(19) United States
(12) Patent Application Publication
(10) Pub. No.: US 2020/0360547 A1
(43) Pub. Date: Nov. 19, 2020

(54) MODULAR WOUND DISINFECTION SYSTEM AND METHOD USING NON-IONIZING ELECTROMAGNETIC RADIATION

(71) Applicants: Robert SMITH, Temple, TX (US); Alan GLOWCZWSKI, College Station, TX (US); Mitchell GREENBERG, Austin, TX (US); Kristen MAITLAND, College Station, TX (US); April LOVELADY, Crockett, TX (US)

(72) Inventors: Robert SMITH, Temple, TX (US); Alan GLOWCZWSKI, College Station, TX (US); Mitchell GREENBERG, Austin, TX (US); Kristen MAITLAND, College Station, TX (US); April LOVELADY, Crockett, TX (US)

(21) Appl. No.: 15/931,267

(22) Filed: May 13, 2020

Related U.S. Application Data
(60) Provisional application No. 62/846,907, filed on May 13, 2019.

(51) Int. Cl. A61L 2/00; A61L 2/24; A61N 5/06

(52) U.S. Cl. CPC A61L 2/0047; A61L 2/24; A61L 2/0052; A61N 5/0624; A61N 2005/0632; A61L 2202/14; A61N 2005/0652; A61N 2005/0628; A61L 2202/11

(57) ABSTRACT

A method for applying LED light treatment includes initiating operation of at least one LED of an LED array of an LED module for a predetermined period of time; and during the predetermined period of time, receiving a signal from a light sensor that indicates light reflected from a surface of a patient's wound. During operation, when the signal from the light sensor indicates that an appropriate dose of light is not being irradiated, operation of the at least one LED is adjusted and when the signal from the light sensor indicates the appropriate dose of light is being irradiated, operation of the at least one LED continues until the predetermined period of time has been achieved.

### CROSS-REFERENCE TO RELATED APPLICATION

This application claims the benefit of U.S. Provisional Application Ser. No. 62/846,907, filed May 13, 2019.

### BACKGROUND

Healthcare associated infections are a major problem in the healthcare industry. When a surgical patient contracts a healthcare associated infection at a hospital, the patient usually requires a longer stay in the hospital because the recovery time is increased, resulting in a large increase in the cost to the hospital and the patient. Some patients, such as the elderly and small children, are more prone to the risk of infection in a hospital.

Bacterial infections are the most common healthcare associated infections. Bacteria can increase recovery time for not only surgical patients but also any patient who has non-intact skin, such as wounds, burns, and ulcers. Therefore, it is important to prevent bacterial infections by providing effective treatment to non-intact skin both in and out of hospitals.

### BRIEF SUMMARY

Modular wound disinfection systems and methods using non-ionizing electromagnetic radiation are provided. Non-intact skin (e.g., a wound) can be treated using a modular wound disinfection system and method using non-ionizing electromagnetic radiation from, for example, light emitting devices (LEDs) of the modular wound disinfection system to inhibit bacterial infections. Non-ionizing radiation refers to radiation that has insufficient energy to cause ionization. That is, the radiation applied by the described systems is not intended to remove an electron from an atom or molecule; however, heat may be generated.

A modular wound disinfection system can be a modular system for applying LED treatment to a wound of a patient. In some cases, a modular system for applying LED treatment to a wound of a patient includes a plurality of LED modules. Each of the LED modules includes at least one LED and allows for consecutive abutment with another LED module. The consecutive abutment may be possible with respect to one or more sides of the LED module. The system also includes a controller coupled to the plurality of LED modules and that controls operation of each LED of the plurality of LED modules.

In some cases, the system includes an electrical connector strip. In such implementations, each of the plurality of LED modules has an electrical terminal so that the electrical connector strip can couple the controller to the plurality of LED modules via attachment to the electrical terminal on each of the plurality of LED modules. In some cases, each of the plurality of LED modules further includes an attachment mechanism that, in operation with a corresponding mechanism at the electrical connector strip or simply in contact with a region of the electrical connector strip, provides secure contact of the electrical terminal for that LED module to the electrical connector strip. In some cases, an LED module includes a wireless communication device to couple to the controller.

In some cases, each of the plurality of LED modules further includes one or more sensors. The one or more sensors can include, but are not limited to, a skin-contact sensor, a light sensor, a temperature sensor, and a combination thereof. The controller can use the feedback from the one or more sensors to ensure that the proper dose is able to be applied.

The skin-contact sensor can be used for sensing whether an LED module is in an appropriate position with respect to the patient's skin. The controller can receive a signal from the skin-contact sensor (e.g., via the electrical connector strip or via the wireless communication device) to determine whether the LED module is in the appropriate position and to cause that LED module to turn off when that LED module is not in the appropriate position to emit the light to the surface of the patient's wound. This automatic turn-off can help avoid light accidentally hitting the patient's eyes (or the eyes of another person in the vicinity). In some cases, the skin-contact sensor is a resistive sensor. In some cases, the skin-contact sensor is a capacitive sensor. In some cases, the skin-contact sensor senses position of the module (with respect to the patient's skin) based on a magnetic field created by a magnetic adhesive that can be provided for releasable attachment of an LED module to a bandage.

The light sensor can be part of the LEDs of the LED module. The light sensor can be used for sensing whether an appropriate amount/dose (e.g., illuminance or irradiance) of light is being irradiated. In some cases, the light sensor can be used to detect whether the LED module is in appropriate position. In some cases, an infrared or ultrasonic sensor may be provided. Such a sensor can be used to confirm distance between the LED module and the skin surface.

The temperature sensor can be used to detect the temperature of the LEDs of the LED module. The controller can receive the temperature information from the temperature sensor and determine whether the temperature of the LEDs of the LED module is within an appropriate temperature range (or in some cases below a particular threshold). In some cases, the temperature can be correlated to energy output of the LEDs (and therefore be used to adjust the power to the LEDs and the corresponding dosage). In some cases, the temperature sensor is used as a safety mechanism to ensure that the temperature does not go to unsafe levels.

A method for applying LED light treatment to a wound of a patient can include initiating operation of an LED system, receiving an indication of a number of LED modules coupled via an electrical connector strip to a controller, determining an appropriate current and voltage according to the number of LED modules, determining dose parameters for a therapy based on received input parameters, and controlling operation of the LED modules according to the dose parameters.

Treatment can be delivered according to the therapy. One or more of the described modular systems delivers the appropriate dose of LED light. In some cases, the method includes operating the LEDs for a predetermined period of time. The predetermined period of time may be predetermined based on the type of the patient's wound. Sensors of the modular system provide feedback mechanisms to indicate whether the appropriate dose was applied as well as to trigger safety measures such as stopping/interrupting application of the LED treatment. In some cases, the method further includes determining whether one or more safety criteria is satisfied. In some cases, the method further includes in response to the safety criteria not being satisfied, ceasing operation of the LED system, and in response to the safety criteria being satisfied, continuing operation until the therapy is complete. In some cases, the safety criteria include temperature and positioning. For example, in some cases, the method includes receiving a measurement of temperature from a temperature sensor; and, in response to the measured temperature being above a predetermined temperature, ceasing operation of one or more of the LED modules. In some cases, this ceasing of operation can help avoid a burn or other harm to the patient. As another example, in some cases, the method includes receiving a signal from a sensor indicating that an LED module is not in position (e.g., via a skin-contact sensor or a reflectance sensor); and in response to receiving the signal indicating the LED module is not in position, ceasing operation of the one or more LED modules. This ceasing of operation can help avoid accidental light irradiation in a person's eyes.

An LED module suitable for the described modular systems for providing LED light treatment to a wound of a patient can include an array of LEDs and a package body that allows for consecutive abutment with other ones of the LED modules and electrical attachment to an electrical connector. In addition, the LED module includes one or more electrical terminals exposed at a surface opposite the direction of irradiation and which are in position to electrically connect to the electrical connector when the LED module is attached to the electrical connector.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A and 1B illustrate example modular wound disinfection systems for delivering LED treatment to a wound of a patient.

FIG. 1C illustrates additional detail of communication in a modular wound disinfection system such as shown in FIGS. 1A and 1B.

FIGS. 2A and 2B illustrate example modular wound disinfection systems having LED modules with an embedded controller.

FIGS. 3A-3C illustrate example configurations of components and sub-components in specific examples of modular wound disinfection systems delivering LED treatment to a wound of a patient.

FIGS. 4A-4C illustrate an example application of an electrical connector strip.

FIG. 5A illustrates a bottom side of an example LED module.

FIG. 5B illustrates an example top side of an LED module.

FIGS. 5C-5E illustrate example side-views of an LED module.

FIGS. 5F and 5G illustrate top sides of example LED modules with indicators.

FIG. 5H illustrates example indicators for an electrical connector strip.

FIGS. 6A-6G illustrate an example application of a modular wound disinfection system.

FIGS. 7A and 7B illustrate an example of a modular wound disinfection system being applied on a wound of a patient.

FIG. 8 illustrates an example of an array of LEDs.

FIGS. 9A and 9B illustrate an example operation of the array of LEDs of FIG. 8.

FIGS. 10A and 10B illustrate example control signals for an operation of the array of LEDs of FIG. 8.

FIG. 11 illustrates a specific example method of applying LED treatment to a wound of a patient.

FIG. 12 illustrates a specific example method of applying LED treatment to a wound of a patient.

FIGS. 13A-13E illustrate a specific example of a modular LED disinfection system on a catheter of a patient.

### DETAILED DESCRIPTION

Modular wound disinfection systems and methods using non-ionizing electromagnetic radiation are provided. Non-intact skin (e.g., a wound) can be treated using a modular wound disinfection system and method using non-ionizing electromagnetic radiation from, for example, LEDs of the modular would disinfection system to inhibit bacterial infections.

Non-ionizing radiation refers to radiation that has insufficient energy to cause ionization. That is, the radiation applied by the described systems is not intended to remove an electron from an atom or molecule; however, heat may be generated.

A modular wound disinfection system can be a modular system for applying LED treatment to a wound of a patent. The system is considered a modular system because certain components of the system are independently usable and able to be configurable on an as-needed basis such that more or fewer components are easily assembled. The modular wound disinfection system can be provided as a kit in which the components are packaged together. In some cases, certain components of the modular wound disinfection system are packaged separately and may even incorporate existing medical supplies or equipment.

A modular wound disinfection system can include at least a controller component and a plurality of LED modules. The LED modules may be packaged together. In some cases, LED modules are be reusable (or at least portions of the LED module are reusable). The reusable portions may be cleaned/disinfected/sterilized according to applicable hospitable standards. When being used to apply LED treatment to a wound, one or more of the LED modules can be applied. The LED modules support the modular capability of the system by being interchangeable and configurable on an as-needed basis. The controller may be a separate component or incorporated as part of one or more of the LED modules.

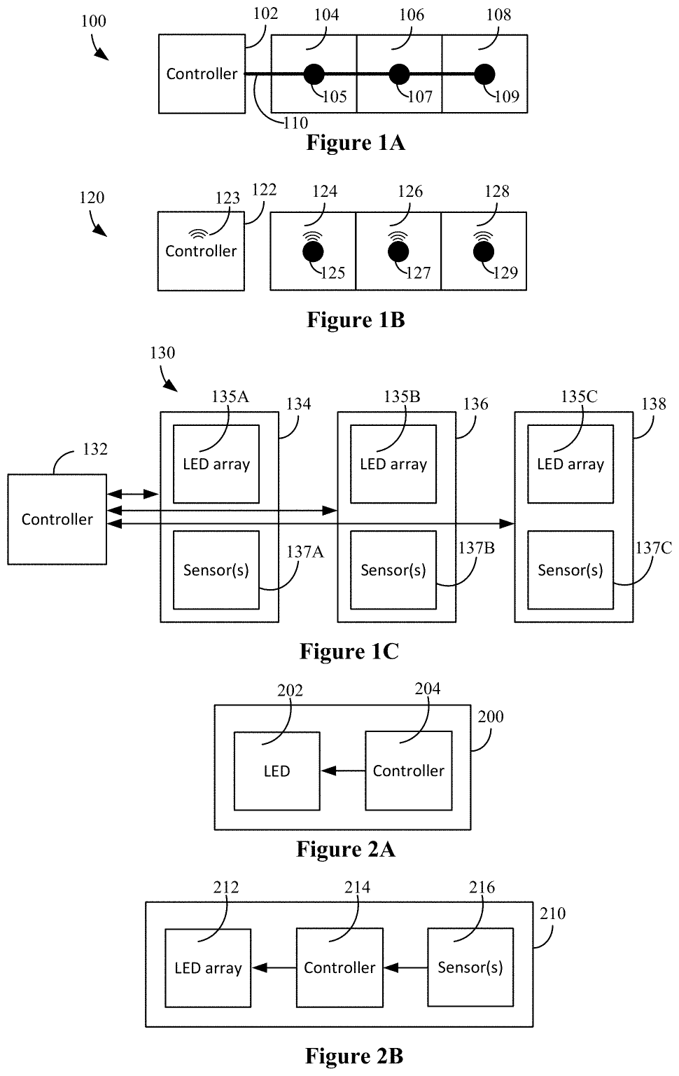

FIGS. 1A and 1B illustrate example modular wound disinfection systems for delivering LED treatment to a wound of a patient. In FIG. 1A, the modular wound disinfection system 100 includes a controller 102 coupled to a plurality of LED modules 104, 106, and 108 through the plurality of LED modules' 104, 106, and 108 corresponding electrical terminals 105, 107, and 109 by way of an electrical connector strip 110. The electrical connector strip 110 provides a conduit of electrical power to the plurality of LED modules 104, 106, and 108 (for implementations in which on-chip power is not provided for the LED modules) as well as communication between the plurality of LED modules 104, 106, and 108 and the controller 102. In some cases, the electrical connector strip 110 provides a conduit of electrical power to all of the devices in the system, including the controller 102. In some cases, the electrical connector strip 110 is a conduit of electrical power to the LED modules 104, 106, and 108 as provided by the controller 102. The electrical connector strip 110 can be of any suitable length and may include a plurality of connections that may be used as needed when configuring the LED modules. When a kit is provided for the system, one or more electrical connector strips having different lengths and widths may be provided. As can be seen in FIGS. 1A and 1B, the plurality of LED modules 104, 106, and 108 are arranged in consecutive abutment.

As used herein, placement in consecutive abutment means that the LED modules are positioned in contact with one another in such a way that the LED modules do not change position in a plane parallel to the surface of the patient's skin. This helps provide coverage of a wound regardless of wound size while using one or more modules. Consecutive abutment may be accomplished through a variety of attachment mechanisms to secure the LED modules in consecutive abutment or may be held in place in at least one plane by friction and/or shape.

In FIG. 1B, the modular wound disinfection system 120 includes a controller 122 and a plurality of LED modules 124, 126, and 128. In this example, the controller 122 includes a wireless communication device 123 that can wirelessly communicate with the plurality of LED modules 124, 126, and 128 through the plurality of LED modules' corresponding wireless communication devices 125, 127, 129. In this example, electrical power may be provided through a battery, electrical cord, electrical connector strip (that would couple to a power source), or some other means of providing electrical power. As can be seen, the plurality of LED modules 124, 126, and 128 are placed in consecutive abutment for use.

FIG. 1C illustrates additional detail of communication in a modular wound disinfection system such as shown in FIGS. 1A and 1B. In the example illustrated in FIG. 1C, a modular wound disinfection system 130 includes a controller 132 in communication with a plurality of LED modules 134, 136, 138. Controller 132 can identify the number of LED modules (e.g., three LED modules 134, 136, 138) that are being connected and may independently communicate with each LED module. However, in some cases, the LED modules 134, 136, 138 can communicate with each other and/or pass signals from the controller 132 between them. Each of the plurality of LED modules 134, 136, 138 includes an LED array 135A, 135B, 135C. In some cases, each of the plurality of LED modules 134, 136, 138 include sensor(s) 137A, 137B, 137C. The sensor(s) 137A, 137B, 137C communicate with the controller 132 to provide information that is used by the controller 132 to control the plurality of LED modules 134, 136, 138. System 130 may be configured such as described with respect to systems 100 and 120 of FIGS. 1A and 1B.

FIGS. 2A and 2B illustrate example representations of LED modules for a modular wound disinfection system with an embedded controller. In some cases, the LED modules 200, 210 may take the place of the controller 102 and one or all of the LED modules 104, 106, 108 as described with respect to FIG. 1A or take the place of the controller 122 and one or all of the LED modules 124, 126, 128 as described with respect to FIG. 1B. In a wired communication configuration, an LED module with an embedded controller may communicate with other LED modules (e.g., without an embedded controller) via an electrical connector. In a wireless communication configuration, the LED module with the incorporated controller may communicate with the other LED modules for control of the other modules. In some cases, each LED module includes an embedded controller, which may support independent control of each of the LED modules.

In FIG. 2A, the LED module 200 includes at least one LED 202 and a controller 204 for controlling the at least one LED 202. In this example, the controller 204 is configured to send signals to control the at least one LED 202, however, other cases may include the ability for the at least one LED 202 to send signals back to the controller 204 (e.g., when one of the at least one LED 202 is used as a light sensor).

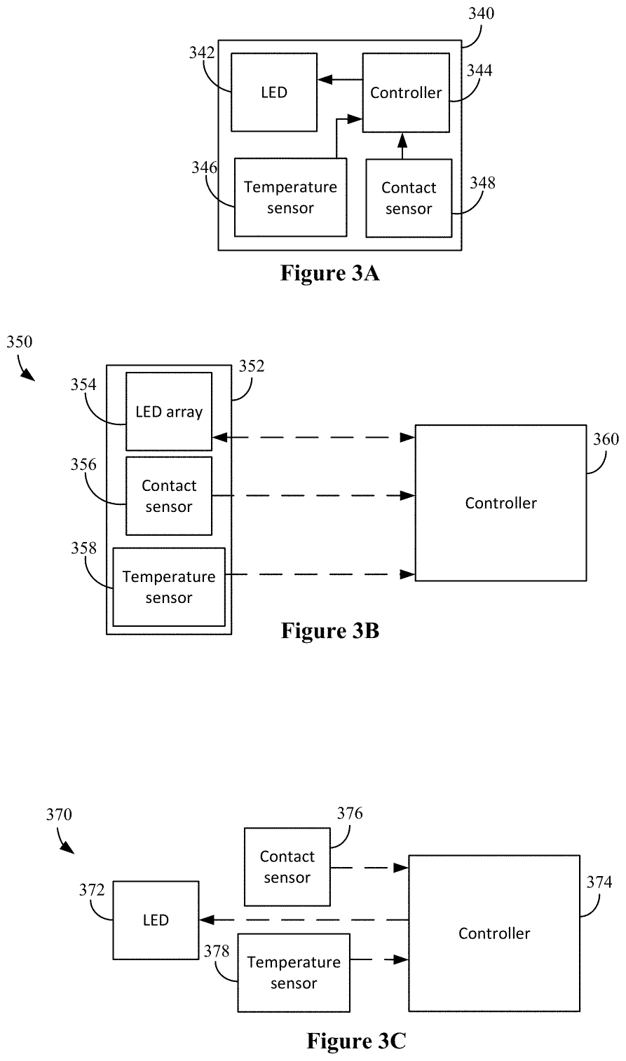

In FIG. 2B, the LED module 210 includes an LED array 212, a controller 214, and a sensor(s) 216. In this example, the sensor(s) 216 send signals to the controller 214. In some examples, the controller 214 may also send signals to the sensor(s) 216 (e.g., to take a measurement). The sensor(s) may be used by the controller 214 to detect conditions such as whether the LED module 210 is in position to emit light to a surface of a patient's skin, whether light is actually being emitted to the surface of the patient's skin (e.g., an infrared or ultrasonic sensor that confirms distance between the LED module 210 and the patient's skin), and/or a temperature between the LED module 210 and the surface of the patient's skin (e.g., the temperature of the LEDs). In some cases, the sensor that detects whether light is actually being emitted to the surface of the patient's skin is at least one LED of the LED array 212, and therefore the LED array 212 would not only receive signals from the controller 214, but send signals to the controller 214 as well. It should be understood that sensors such as described with respect to FIG. 2B may be integrated with any LED module (with or without integrated controller) or provided as a separate component that can be coupled to the system (and controller). Examples of various configurations of modular wound disinfection systems incorporating the sensors are shown in FIGS. 3A-3C.

FIGS. 3A-3C illustrate example configurations of components and sub-components in specific examples of modular wound disinfection systems delivering LED treatment to a wound of a patient. FIG. 3A provides an example implementation of LED module 210 of FIG. 2B. In the example illustrated in FIG. 3A, an LED module 340 includes at least one LED 342, a controller 344, a temperature sensor 346, and a contact sensor 348. In this example, the LED module 340 may not need any other components to deliver LED treatment to a wound of a patient and supports small area wounds by itself. The temperature sensor 346, which measures the temperature between the LED module 340 and a surface of a patient's wound, and the contact sensor 348, which is used to determine whether the at least one LED 342 of the LED module 340 is in position to emit light to the surface of the patient's wound, can send signals containing these measurements to the controller 344 to so that the controller 344 can determine whether changes need to be made to the settings (e.g., current, voltage, length of time) for the LED module 340 to emit light to the surface of the patient's wound and/or be used by the controller 344 as a safety feature (e.g., to stop the at least one LED 342 from emitting light when a predetermined temperature is reached/exceeded and/or stop the at least one LED 342 from emitting light when the at least one LED 342 of the LED module 340 is not in position to emit light to the surface of the patient's wound because the light emitted directly into a person's eyes may be harmful). In some cases, one or both of the temperature sensor 346 and contact sensor 348 may be located off-module (and coupled separately to the controller 344).

In FIG. 3B, a modular wound disinfection system 350 includes at least an LED module 352 with in-module sensor components 356, 358 and a controller 360. As illustrated in FIG. 3B, the LED module 352 includes an LED array 354, a contact sensor 356, and a temperature sensor 358. In this example, the LED array 354 can also act as a light sensor as well as provide light at the surface of the patient's wound. In some cases, one or both of the temperature sensor 358 and contact sensor 356 may be located off-module (and coupled separately to the controller 360).

For example, in the illustrative example of FIG. 3C, a modular wound disinfection system 370 includes separate sensor components. Here, the wound disinfection system 370 includes at least an LED module 372, a controller 374, a contact sensor 376, and a temperature sensor 378.

As previously mentioned, such as described with respect to FIG. 1A, in some cases, a modular wound disinfection system includes an electrical connector strip. In such implementations, each of the plurality of LED modules has an electrical terminal so that the electrical connector strip can couple the controller to the plurality of LED modules via attachment to the electrical terminal on each of the plurality of LED modules. In some cases, each of the plurality of LED modules further includes an attachment mechanism that, in operation with a corresponding mechanism at the electrical connector strip or simply in contact with a region of the electrical connector strip, provides secure contact of the electrical terminal for that LED module to the electrical connector strip.

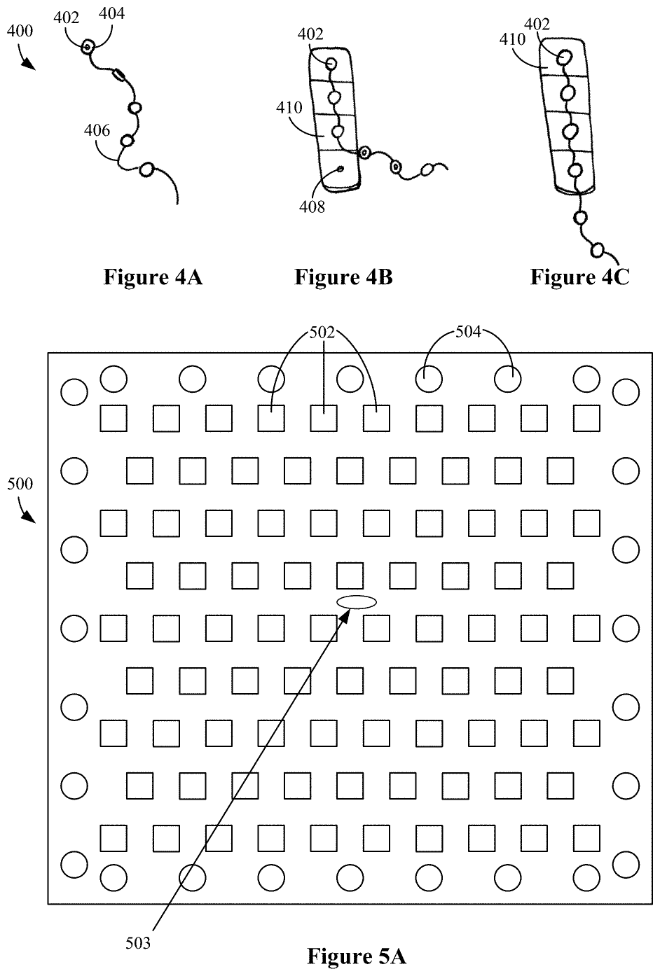

FIGS. 4A-4C illustrate an example application of an electrical connector strip. Referring to FIG. 4A, an electrical connector strip 400 can be provided that includes an electrical wire or cord 406 (or tape or other structure that can include one or more conductive lines) with a plurality of electrical terminals 402 and corresponding attachment mechanisms 404. The cord 406 can contain a plurality of conductive lines where one or more of the electrical terminals 402 are provided for each of the conductive lines. In some cases, such as where a same signal is provided to each LED module, a single conductive line may be provided for supplying power to all of the LED modules. In some cases, each LED module couples to its own conductive line via the corresponding electrical terminal 402 of the electrical connector strip. The electrical connector strip thus can couple each LED module to a controller.

As can be seen in FIGS. 4B and 4C, any number of LED modules 410 can be arranged in consecutive abutment and coupled to a controller using the electrical connector strip 400. The electrical connector strip 400 can have a certain number of terminals/attachment mechanisms and not all of the available terminals/attachment mechanisms need to be used.

In some cases, the electrical terminals 402 of the electrical connector strip 400 may be a male-ended plug, a female ended jack, or some variation between a male-ended plug and a female-ended jack, depending on the configuration of the electrical terminals 408 of the LED modules 410. For example, if electrical terminals 408 of the LED modules 410 are provided with an attachment mechanism in the form of female-ended jacks, the corresponding attachment mechanisms 404 of the electrical connector strip 400 will be male-ended plugs. Conversely, if electrical terminals 408 of the LED modules 410 are provided with an attachment mechanism in the form of male-ended plugs, the attachment mechanisms 404 of the electrical connector strip 400 will be female-ended jacks. Other examples of attachment mechanisms (for LED module and/or electrical connector strip) include, but are not limited to hybrid male/female terminals.

In some cases, the electrical connector strip 400 provides communication between the plurality of LED modules 410 and a controller as well as power. In some cases, such as those in which a controller is included within each individual LED module 410, the electrical connector strip 400 may only provide power to the plurality of LED modules 410. In some cases, the electrical strip connector 400 may also provide a means for communicating between controllers. In some cases, such as those in which a battery is provided with each of the plurality of LED modules 410, the electrical strip connector 400 may only provide a means for communicating between the controllers.

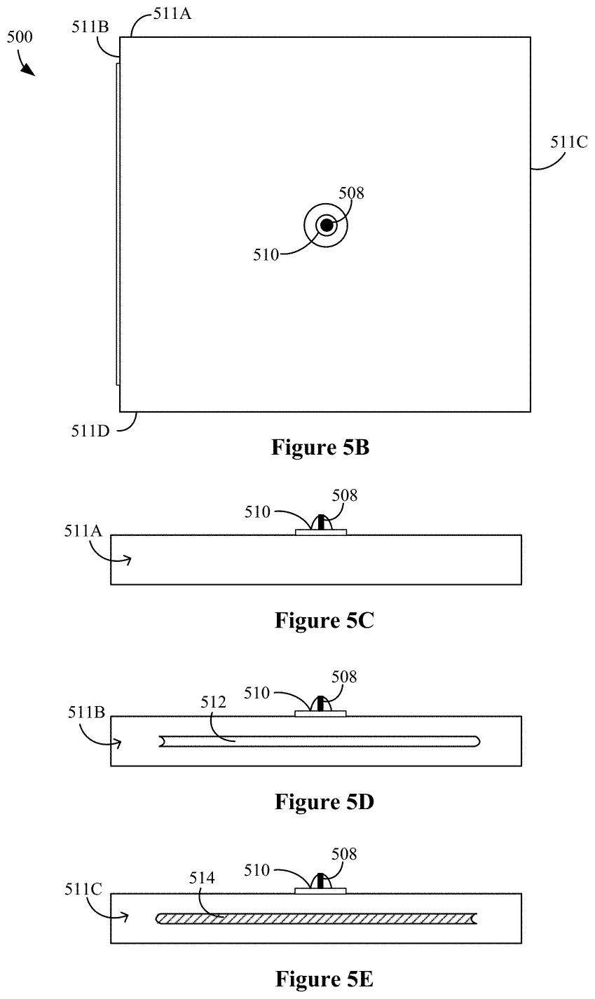

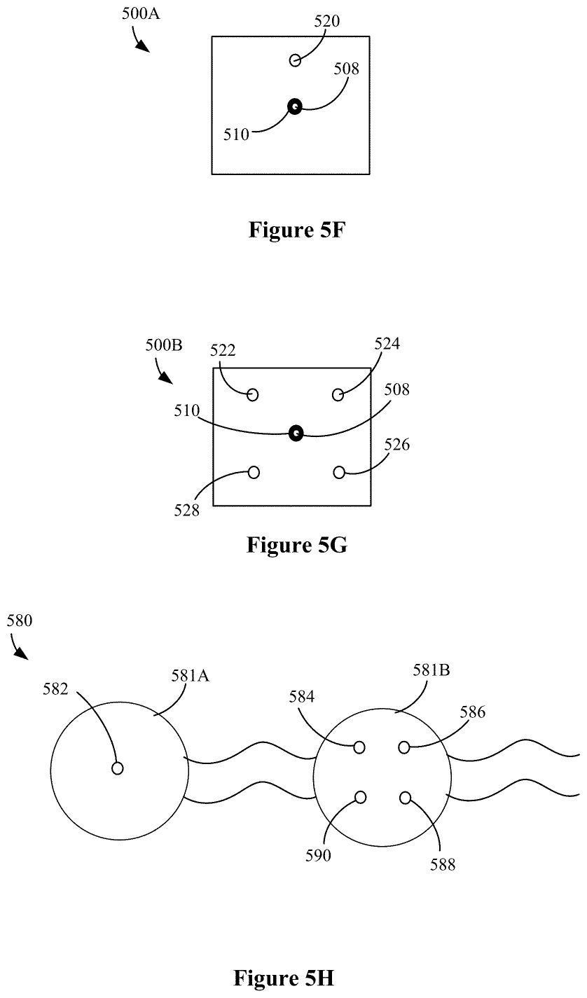

FIG. 5A illustrates a bottom side of an example LED module; FIG. 5B illustrates an example top side of an LED module; and FIGS. 5C-5E illustrate example side-views of an LED module. An LED module suitable for the described modular systems for providing LED light treatment to a wound of a patient can include an array of LEDs and a package body that allows for consecutive abutment with other ones of the LED modules and electrical attachment to an electrical connector.

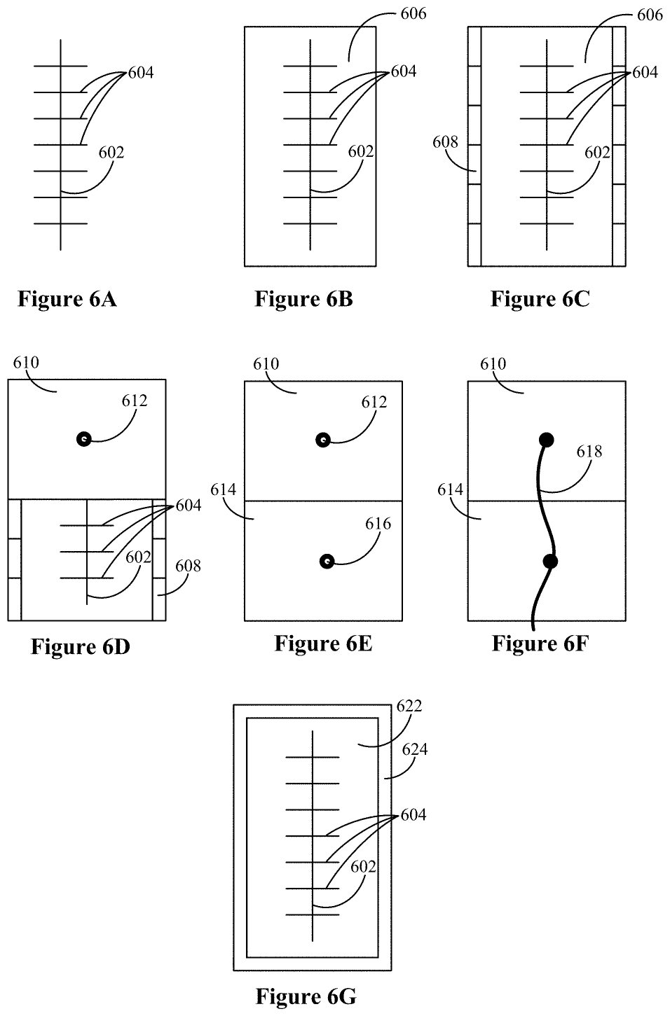

Referring to FIG. 5A, the bottom, or front side, 501 of an LED module 500 may be positioned to face the surface of the patient's wound. In this example, the LED module 500 includes a plurality of LEDs 502 in an array. The number of LEDs 502 in the array can depend on the size of the module 500 as well as the lighting characteristics of the LEDs 502. In some cases, the number of LEDs 502 depends on the material covering the LEDs, which can function as a diffusion layer (not shown). In some cases, a sensor 503 can be disposed between the LEDs 502. In addition, in some cases, a plurality of magnets 504 can be embedded into the LED module 500 near the edges to facilitate attachment to a patient (as will be discussed in more detail with respect to FIGS. 6A-6G). For example, the plurality of magnets 504 can be used to secure the LED module 500 into the correct position over the surface of a patient's wound (e.g., when using magnetic tape around the outer edge of the surface of the patient's wound). In some cases, one or more of the plurality of magnets 504 can be part of a contact sensor. It should be noted that other circuitry (not shown) may be embedded in the packaging of the LED module 500; and the packaging can include a flexible and/or transparent substrate.

Referring FIG. 5B, the top, or away facing (from the patient's wound) side, of the LED module 500 can include one or more exposed electrical terminals (e.g., conductive terminal 508) that are in position to electrically connect to an electrical connector when the LED module is attached to the electrical connector. An attachment mechanism 510 for coupling the LED module 500 to the electrical connector strip (e.g., electrical connector strip 400) can be provided in any suitable form.

In the illustrated example, the attachment mechanism 510 includes a snap attachment (e.g., in the form of an eyelet/post) is shown. In this illustrated example, the package body has a male cylindrical body that is raised in a plane perpendicular to main body of the LED module and a snap fastener around the male cylindrical body, where the one or more electrical terminals 508 are exposed within the male cylindrical body. In some cases, the snap attachment is in the form of a stud or socket. In other cases, the electrical terminal 508 may be in a block shape, with prongs, or any other shape/configuration that is suitable for providing an electrical connection to an LED module. Of course, the attachment mechanism 510 may be any type of attachment mechanism suitable for attaching LED module to an electrical connector such that electrical terminal(s) 508 can make electrical connection to conductive line(s) of the electrical connector. Examples of attachment mechanisms include, but are not limited to, a snap fastener, a spring fastener, a buckle, a button, a screw, a cable tie, a screw and nut, a bolt and nut, a hook and loop fastener, a pin lock, a latch, a threaded rod, and a rivet.

The sides (e.g., sides 511A, 511B, 511C, 511D) of the LED module 500 can be configured for ease of consecutive abutment, for example, as illustrated in FIGS. 5B and 5C, a side 511A can be a flat surface so that other LED modules may directly abut the LED module 500. In some cases, the side(s) can have texture or a releasable, re-stickable, or re-adherable adhesive.

In some cases, the consecutive abutment can be facilitated by an attachment mechanism (e.g., by a pre-tapped hole, screws, and a bar attaching to each LED module placed in consecutive abutment) that is attached to the LED module 500.

For example, as illustrated in FIGS. 5D and 5E an interlocking mechanism 512 that is designed to interlock securely to the corresponding interlocking mechanism 514 can be provided on opposing sides (e.g., side 511B and side 511C).

FIGS. 5F and 5G illustrate top sides of example LED modules with indicators; and FIG. 5H illustrates example indicators for an electrical connector strip. Referring to FIG. 5F, an LED module 500A can include an indicator light 520 at the top, or away facing (from the patient's wound) side, of the LED module 500A in addition to the exposed one or more electric terminals 508 and attachment mechanism 510. The indicator light 520 can indicate whether power is being provided to the LED module 500A and/or whether the LEDs (not shown) on the LED module 500A are emitting light. In some cases, the indicator light 520 can include a plurality of LEDs in the same package such that the indicator light 520 can illuminate with different colors. For example, in a scenario where LED module 500A includes sensors, the indicator light 520 can indicate that all sensors are measuring acceptable readings through a green indicator light, that a temperature sensor is not measuring an acceptable temperature through a different colored indicator light (e.g., a yellow indicator light), that a contact sensor is detecting the LED module 500A is not in position to emit light to a wound of a patient through a different colored indicator light (e.g., a blue indicator light), and/or that a light sensor is not detecting light reflected from a surface of a patient's wound through a different colored indicator light (e.g., an orange indicator light).

Referring to FIG. 5G, instead of a single indicator light, an LED module 500B can include a plurality of indicator lights 522, 524, 526, 528 at the top, or away facing (from the patient's wound) side, of the LED module 500B in addition to the exposed one or more electric terminals 508 and attachment mechanism 510.

Indicator lights 522, 524, 526, 528 of LED module 500B may indicate whether the corresponding sensors are measuring acceptable readings and/or indicate whether the LEDs of the LED module 500B are emitting light based on those readings. Each of the indicator lights 522, 524, 526, 528 of LED module 500B may represent different indications. For instance, indicator light 522 may indicate whether LED module 500B is receiving power or emitting light. In an example implementation, indicator light 522 may indicate, through a red indicator light, that the LEDs of the LED module 500B are not emitting light and indicate through a green indicator light that the LEDs of the LED module 500B are emitting light.

Indicator light 524 may indicate whether a temperature of the LEDs is within a predetermined acceptable range or below a predetermined temperature (e.g., where a temperature above the predetermined temperature may cause injury to the patient). Indicator light 526 may indicate whether the LED module 500B is in position for LEDs to appropriately emit light to the wound of the patient. For instance, indicator light 524 may indicate, through a red indicator light, that the temperature sensor is measuring is too high of a temperature (and it is therefore unsafe to continue to apply LED light treatment from the LEDs of the LED module 500B) and that the LEDs of the LED module 500B are not emitting light. The indicator light 524 may indicate, through a yellow indicator light, that the temperature the temperature sensor is measuring is almost too high (and it is therefore almost unsafe to continue to apply LED light treatment from the LEDs of the LED module 500B) and that the LEDs of the LED module 500B are still emitting light. The indicator light 524 may indicate, through a green indicator light, that the temperature the temperature sensor is measuring an acceptable temperature (and it is therefore safe to continue to apply LED light treatment from the LEDs of the LED module 500B) and that the LEDs of the LED module 500B are emitting light.

The indicator light 526 may indicate, through a red indicator light, that LED module 500B is not in position (e.g., based on a contact sensor) and indicate, through a green indicator light, that the LED module 500B is in position.

The indicator light 528 may indicate, through a red indicator light, that the light sensor is not detecting light from the LEDs of the LED module 500B and indicate, through a green indicator light, that the light sensor is detecting light from the LEDs of the LED module 500B.

Of course, depending on the number of sensors, more or fewer indicator lights can be part of an LED module (e.g., through different colored indicator lights or through more or fewer indicator lights. In addition, in some cases, instead of or in addition to indicator lights at the LED module, one or more of the indicator lights may be on the electrical connector strip (e.g., as illustrated in FIG. 5H). Referring to FIG. 5H, each connector node 581A, 581B of the electrical connector strip 580 can include one or more indicators. The one or more indicators may include an indicator light 582 that is configured to function similar to indicator light 520 of FIG. 5F or may include indicator lights 584, 586, 588, 590 that is configured to function similar to indicator lights 522, 524, 526, 528 of FIG. 5G.

It should be noted that in some cases, each LED module may include a tracking device (e.g., GPS tracking device) so that each LED module can be found and identified. In other words, each LED module may include its own module number and be able to be tracked and found wherever the LED module is located (e.g., within a hospital).

FIGS. 6A-6G illustrate an example application of a modular wound disinfection system. The modular wound disinfection system can be easily applied during the course of conventional bandaging.

FIG. 6A illustrates a state of a wound (e.g., incision) 602 of a patient that has been treated with a plurality of sutures 604. Of course, it should be understood that the described modular wound disinfection system can be applied to wounds that have been treated with stitches and/or staples and/or adhesives and even wounds that have not been sealed (but that at least are covered with a clear bandage).

Referring to FIG. 6B, a clear adhesive bandage 606 can be applied over the wound 602 of the patient. Then, as shown in FIG. 6C, magnetic tape 608 is applied to outer edges of the clear adhesive bandage 606. The magnetic tape 608 may be available with the supplies such as the adhesive bandages 606 or provided in a kit with the LED modules. In some cases, the magnetic tape 608 is a double-sided adhesive instead of a magnetic tape, such that the plurality of magnets 504 described in FIG. 5A are not needed to apply the LED module over the wound 602 of the patient.

Once the magnetic tape 608 is in position, as illustrated by FIGS. 6D and 6E, LED modules 610 and 614 can be positioned/securely attached over the wound 602 of the patient by the attraction between the magnetic tape 608 and a plurality of magnets (e.g., the plurality of magnets 504 described in FIG. 5A) on the LED modules 610, 614. The two LED modules 610, 614 can be placed in consecutive abutment in order to fully cover the wound 602. The LED modules 610, 614 can be configured as described with any of the LED modules shown in FIGS. 1A-5C.

Next, as illustrated in FIG. 6F, an electrical connector strip 618 is coupled to the electrical terminals (e.g., electrical terminal 612 of LED module 610 and electrical terminal 616 of LED module 614 of FIG. 6E). The electrical connector strip 618 can couple the LED modules 610, 614 to a controller (not shown). At this stage, the modular wound disinfection system is ready for use.

FIG. 6G illustrates an alternative bandage that may be applied in place of bandage 606 and magnetic tape 608. Referring to FIG. 6G, in some cases, a clear adhesive bandage 622 may be applied that includes a magnetic frame 624 or adhesive (e.g., for implementations where magnets such as described in FIG. 5A are omitted).

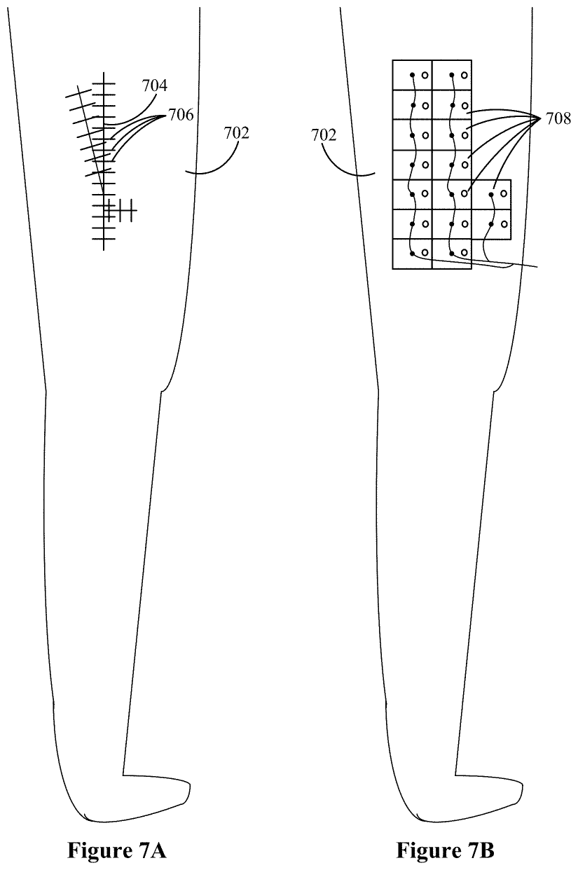

FIGS. 7A and 7B illustrate an example of a modular wound disinfection system being applied on a wound of a patient. It should be noted that the LED modules described in FIG. 7B may be implemented as described with any of the LED modules referred to herein; and that the LED modules may include an internal controller or may be connected (e.g., via an electrical connector strip) to an external controller.

As can be seen in FIG. 7A, a leg 702 of a patient is illustrated having a wound 704 that has been treated with a plurality of sutures 706. In FIG. 7B, a plurality of LED modules 708 are applied to the leg 702 of the patient, covering the wound 704. As can be seen, because the plurality of LED modules 708 are configured for consecutive abutment, some of the plurality of LED modules 708 are placed in consecutive abutment with other LED modules of the plurality of LED modules 708 on four lateral sides of the LED module.

It should be noted that LED modules do not have to be the square shape shown in FIG. 7B; LED modules may be any shape that allows for consecutive abutment, such as triangular, pentagonal, hexagonal, jigsaw puzzle shape (e.g., interlocking tessellating pieces), and so forth.

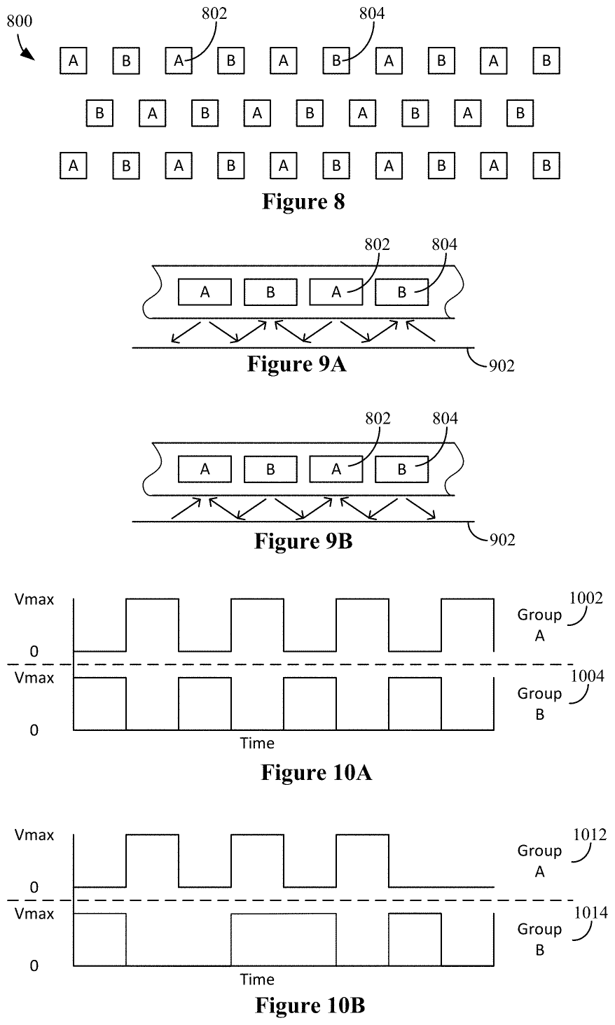

FIG. 8 illustrates an example of an array of LEDs; FIGS. 9A and 9B illustrate an example operation of the array of LEDs of FIG. 8; and FIGS. 10A and 10B illustrate example control signals for an operation of the array of LEDs of FIG. 8.

LEDs in the LED array 800 may be controlled together or in two or more groupings. FIG. 8 illustrates an example of a two-grouping control of the LEDs. A first group 802, indicated by “A,” and a second group 804, indicated by “B,” can be selected in a checkerboard pattern. The two-grouping control is useful for scenarios where one group functions to emit light while the other group functions to detect the light reflecting back. Alternatively, the LEDs of the different groups may be different wavelength LEDs. For example, one group can be blue/violet LEDs and the other can be red LEDs. In some examples, for the blue/violet LEDs, the wavelength range may be between about 380 and 470 nanometers. In some examples, for the blue/violet LEDs, the wavelength range is 405 nanometers.

For the emitting/receiving configuration, FIGS. 9A and 9B illustrate operation of the two-groupings of the LEDs. In FIG. 9A, light is emitted from Group A LEDs 802, reflected off of a surface 902 (e.g., a surface of a wound of a patient), and received by Group B LEDs 804. In FIG. 9B, light is emitted from Group B LEDs 804, reflected off of the surface 902, and received by Group A LEDs 802. When Group A or B LEDs 802, 804 receive light, they may function similar to a photodiode to detect light from the group of LEDs that are emitting the light. The alternating emitting and receiving may be controlled using a signal from the controller that turns on and off each group of the LEDs. Circuitry on the LED module can select the appropriate LEDs in the array to receive the control signal.

Referring to FIG. 10A, the control signal 1002 supplied to Group A can be the inverse of the control signal 1004 supplied to Group B in order to achieve the alternating approach described above. As can be seen, a predetermined length of time that Group A LEDs emit light is the same as a predetermined length of time that Group B LEDs emit light. It should be understood that the predetermined length of time is a length of time that may be changed based on the type and severity of the wound the light is treating.

As can be seen in FIG. 10B, other patterns can be applied. For example, a control signal 1012 applied to Group A LEDs can cause the Group A LEDs to alternate on and off at a constant frequency; whereas, the Group B LEDs may be on and off using a different pattern such as indicated by control signal 1014. Here, Group A LEDs and Group B LEDs sometimes emit light at the same time, sometimes do not emit light at the same time, and sometimes emit light while the other is not emitting light. This may be useful in targeting specific types of bacteria. It should be noted that when both Group A and B LEDs are emitting light, some LEDs may be independently controlled (separate from Group A and Group B) to function as receiver/detectors.

A method for applying LED light treatment to a wound of a patient can include initiating operation of an LED system comprising a plurality of LED modules, receiving an indication of a number of the plurality of LED modules coupled via an electrical connector strip to a controller, determining an appropriate current and voltage according to the number of the plurality of LED modules, determining dose parameters for a therapy based on received input parameters, and controlling operation of the plurality of LED modules according to the dose parameters.

Treatment can be delivered according to the therapy by one or more of the described modular systems. In some cases, the method includes operating the plurality of LEDs for a predetermined period of time. The predetermined period of time may be predetermined based on a type of the patient's wound. Sensors of the modular system provide feedback mechanisms to indicate whether the appropriate dose was applied as well as to trigger safety measures such as stopping application of the LED treatment. In some cases, the method further includes determining whether one or more safety criteria is satisfied. In some cases, the method further includes in response to the safety criteria not being satisfied, ceasing operation of the LED system, and in response to the safety criteria being satisfied, continuing operation until the therapy is complete. In some cases, the safety criteria include temperature and positioning. For example, in some cases, the method includes receiving a measurement of temperature from a temperature sensor; and, in response to the measured temperature being above a predetermined temperature, ceasing operation of one or more of the LED modules. In some cases, this ceasing of operation can help avoid a burn or other harm to the patient. As another example, in some cases, the method includes receiving a signal from a sensor indicating that an LED module is not in position (e.g., via a skin-contact sensor or a reflectance sensor); and in response to receiving the signal indicating the LED module is not in position, ceasing operation of the one or more LED modules. This ceasing of operation can help avoid accidental light irradiation in a person's eyes.

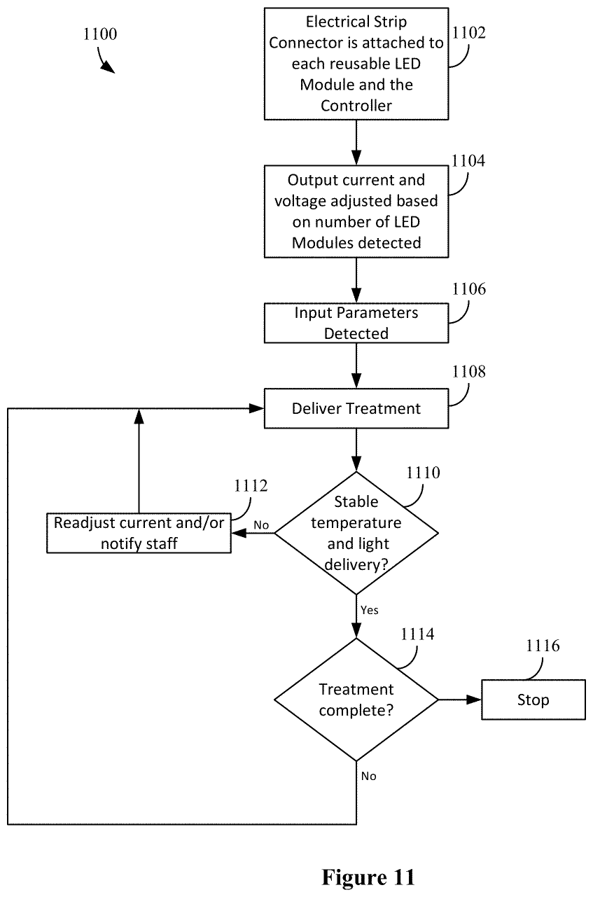

FIG. 11 illustrates a specific example method of applying LED treatment to a wound of a patient. The method 1100 (executed by the controller) may begin, for example, when an electrical connector strip is attached (1102) to each LED module and the controller. In particular, the controller can detect that a certain number of modules are connected. In some cases, the controller may be separate from each LED module. In some cases, a controller is embedded within each LED module. The controller then outputs (1104) the current and voltage that is adjusted based on a number of LED modules detected. In cases in which the controller is embedded within each LED module, this step may be predetermined and/or unneeded. Input parameters may be detected/obtained (1106). The input parameters may include the type and size of a wound of a patient, the type of bacteria that is trying to be destroyed, the sensitivity of the patient's skin to LED light, and the number of treatments of LED light that may be needed, among other parameters. The parameters may also include the length of time for each treatment or the length of time may be calculated based on the other parameters received.

The treatment is delivered (1108) to the wound of the patient via the LED modules under the control of the controller. In this example, the controller also determines (1110) whether a temperature of the LEDs is stable/acceptable and whether the light is being delivered correctly. As previously discussed, this determination may be made from information received by the controller from temperature sensors, light sensors, and/or contact sensors. If the controller determines that any of these factors are unacceptable, the controller readjusts (1112) the current and/or notifies the staff (e.g., through an indicator light) of the unacceptable factors so that treatment may continue to be delivered (1108) to the wound of the patient. While the controller determines that all of these factors are acceptable, the controller may determine (1114) whether the LED light treatment is complete. This determination may be a time-based determination, which may be based on the input parameters, or based on some other measured determination (e.g., the intensity of the LED light being emitted for the type of wound of the patient). If the controller determines that the LED light treatment is complete, the process is stopped (1116). If the treatment is not complete, the process returns to continue delivering (1108) treatment to the wound of the patient.

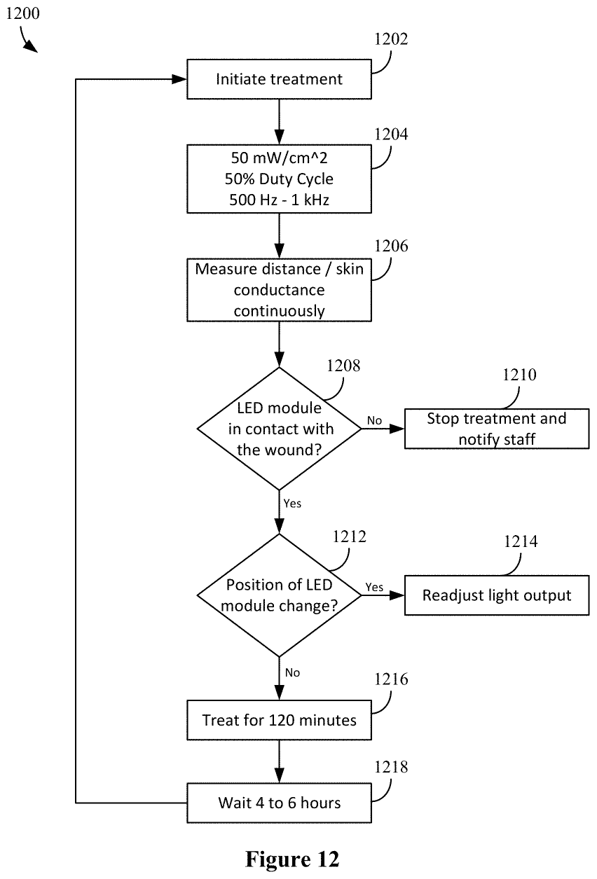

FIG. 12 illustrates a specific example method of applying LED treatment to a wound of a patient. In this example method 1200, the treatment is initiated (1202) by using (1204) predetermined parameters. The predetermined parameters may be standard for every LED module, or may be predetermined by the controller based on the number of LED modules detected, the type of wound and/or the size of the wound of the patient, and/or length of treatment time, among other things. In this example, the preferred predetermined parameters are 50 mW/cm\hat{ }2, 50% Duty Cycle, and 500 Hz-1 kHz.

During treatment, sensors may be continuously checked for acceptable readings/measurements. In other cases, the sensors may be periodically checked. As can be seen in this example, the distance between the LED module and the wound of the patient is measured (1206) continuously, as is the skin conductance. The LED module is also checked (1208) continuously to ensure the LED module is in contact with the wound of the patient. This may be accomplished by a contact sensor. If the LED module is not in contact with the wound of the patient, treatment is stopped (1210) and/or staff is notified. The position of the LED module is also checked (1212) for a change. This may be accomplished through a light sensor. If the position of the LED module is changed, the light output of the LEDs is readjusted (1214). In this example, as long as acceptable readings from the sensors are measured, the LED modules will treat (1216) the wound of the patient for 120 minutes. At the end of the 120 minutes, the controller will shut down the LED modules and wait (1218) 4 to 6 hours before initiating (1202) treatment again. It should be understood that the length of time for treatment and the shutdown are specific examples and other implementations may utilize different lengths of time.

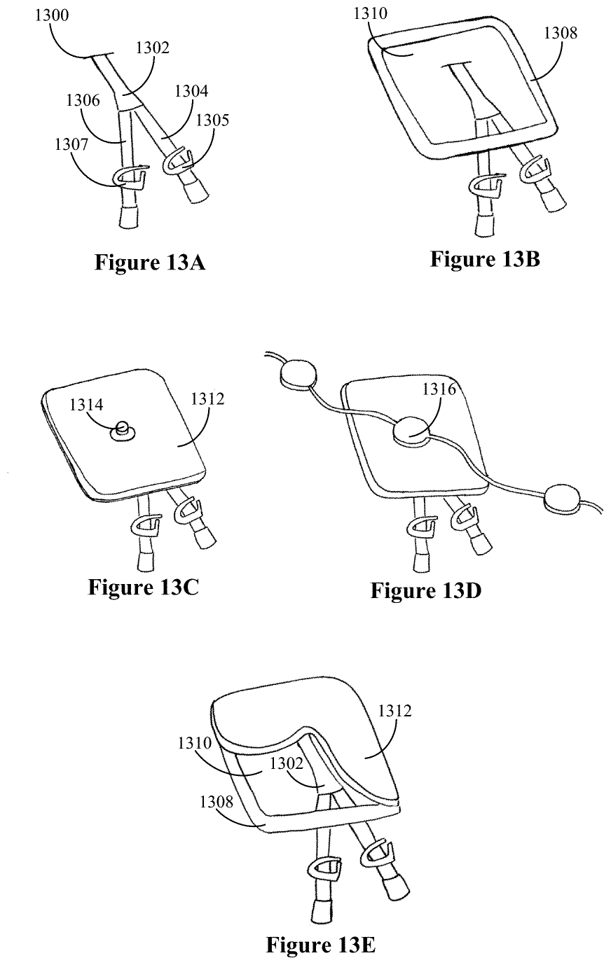

FIGS. 13A-13E illustrate a specific example of a modular LED disinfection system on a catheter of a patient. Catheters have a high risk of causing infection because parts of the catheter are exposed outside a patient's body. The described modular LED disinfection system can be used on “wounds” such as incisions made for a catheter to reduce infections. Referring to FIG. 13A, an incision 1300 is illustrated, from which a catheter 1302 having a first lumen 1304 and corresponding clamp 1305, and a second lumen 1306 and corresponding clamp 1307 is exposed. In FIG. 13B, a clear adhesive bandage 1310 and magnetic or adhesive outer edge 1308 are illustrated. The outer edge 1308 may be made of magnetic tape for magnetic attraction with an LED module or may simply be an adhesive.

Referring to FIG. 13C, an LED module 1312 can be positioned on the clear adhesive bandage 1310 and attached via the outer edge 1308 described with respect to FIG. 13B. The LED module 1312 includes an attachment mechanism 1314 such that, as shown in FIG. 13D, an electrical strip connector 1316 can be attached to the LED module. Referring to FIG. 13E, by using the magnetic outer edge 1308 or a releasable adhesive outer edge 1308, the LED module 1312 may be peeled back so that a technician/nurse/doctor/hospital staff may easily check the incision 1300 and catheter 1302 of the patient. This is advantageous because, if the treatment of the incision 1300 is working, the system allows the technician/nurse/doctor/hospital staff to check for that and simply put the LED module 1312 back into position without changing the clear adhesive bandage 1310. This provides an advantage over other systems and methods of treating wounds and incision of patients because those generally use some sort of cream that may not allow a technician/nurse/doctor/hospital staff to see how the wound/incision looks (i.e., to check for infection) without removing the bandage.

A method for applying LED light treatment to a wound of a patient can, at a minimum, include initiating operation of at least one LED of an LED array of an LED module for a predetermined period of time; during the predetermined period of time, receiving a signal from a light sensor that indicates light reflected from a surface of the patient's wound; in response to the signal from the light sensor indicating that an appropriate dose of light is not being irradiated, adjusting operation of the at least one LED of the LED array of the LED module; and in response to the signal from the light sensor indicating the appropriate dose of light is being irradiated, continue operation of the at least one LED of the LED array of the LED module for the predetermined period of time.

In some cases, the method further includes receiving a signal from a skin-contact sensor that indicates whether the LED module is in position for the LED array of the LED module to emit light to the surface of the patient's wound, wherein the initiating operation of the at least one LED of the LED array of the LED module is performed in response to the signal from the skin-contact sensor indicating that the LED module is in position for the LED array of the LED module to emit the light to the surface of the patient's wound. Here, a controller performing the method can cease operation of the at least one LED of the LED array of the LED module in response to receiving a subsequent signal from the skin-contact sensor indicating that the LED module is not in the position.

In some cases, the method alternatively or further includes receiving a measurement of temperature from a temperature sensor; and in response to the measured temperature being above a predetermined temperature, ceasing or adjusting operation of the at least one LED of the LED array of the LED module.

Kits for use in practicing certain methods described herein are also provided. In certain embodiments, a kit may include one or more of: a plurality of LED modules, one or more lengths of electrical connector strips, clear adhesive bandages, magnetic tape, and, in certain cases, a computer-readable storage medium that when executed by a processor of a controller can perform any of the methods described above for dosing and controlling the LED modules. In certain embodiments, the kits will further include instructions for practicing the subject method or means for obtaining the same (e.g., a website URL directing the user to a webpage which provides the instructions), where these instructions may be printed on a substrate, where substrate may be one or more of: a package insert, the packaging, containers and the like. In the subject kits, the one or more components are present in the same or different containers, as may be convenient or desirable. In an example implementation, a kit includes a plurality of LED modules; and one or more lengths of electrical connector strips; and, optionally, clear adhesive bandages; and, optionally, magnetic tape. An LED module for providing LED light treatment to a wound of a patient (and which can be included in the kit) can include an array of LEDs; and a package body that allows for consecutive abutment with at least one other LED module and electrical attachment to an electrical connector. Such an LED module can further include one or more electrical terminals exposed at a surface of the package body on an opposite side to a side providing irradiation and which are in position to electrically connect to the electrical connector when the LED module is attached to the electrical connector.

It should be understood that as used herein, in no case do the terms “storage media,” “computer-readable storage media” or “computer-readable storage medium” consist of transitory carrier waves or propagating signals.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A method for applying LED light treatment to a wound of a patient, comprising:
initiating operation of at least one LED of an LED array of an LED module for a predetermined period of time;
during the predetermined period of time, receiving a signal from a light sensor that indicates light reflected from a surface of a patient's wound;
in response to the signal from the light sensor indicating that an appropriate dose of light is not being irradiated, adjusting operation of the at least one LED of the LED array of the LED module; and
in response to the signal from the light sensor indicating the appropriate dose of light is being irradiated, continue operation of the at least one LED of the LED array of the LED module for the predetermined period of time.

2. The method of claim 1, further comprising ceasing operation of the at least one LED of the LED array of the LED module after the predetermined period of time has expired.

3. The method of claim 1, wherein the predetermined period of time is predetermined based on a type of the patient's wound.

4. The method of claim 1, further comprising:
receiving a measurement of temperature from a temperature sensor; and
in response to the measured temperature being above a predetermined temperature, ceasing or adjusting operation of the at least one LED of the LED array of the LED module.

5. The method of claim 1, further comprising:
receiving a signal from a skin-contact sensor that indicates whether the LED module is in position for the LED array of the LED module to emit light to the surface of the patient's wound,
wherein the initiating operation of the at least one LED of the LED array of the LED module is performed in response to the signal from the skin-contact sensor indicating that the LED module is in position for the LED array of the LED module to emit the light to the surface of the patient's wound.

6. The method of claim 5, further comprising ceasing operation of the at least one LED of the LED array of the LED module in response to receiving a subsequent signal from the skin-contact sensor indicating that the LED module is not in the position.

7. A method for applying LED light treatment to a wound of a patient comprising:
initiating operation of an LED system comprising a plurality of LED modules;
receiving an indication of a number of the plurality of LED modules coupled via an electrical connector strip to a controller;
determine an appropriate current and voltage according to the number of the plurality of LED modules;
determining dose parameters for a therapy based on received input parameters; and
controlling operation of the plurality of LED modules according to the determined dose parameters.

8. The method of claim 7, further comprising delivering treatment according to the therapy.

9. The method of claim 7, further comprising determining whether one or more safety criteria is satisfied.

10. The method of claim 9, wherein in response to the safety criteria not being satisfied, ceasing operation of the LED system, wherein in response to the safety criteria being satisfied, continuing operation of the LED system until the therapy is complete.

11. A system for applying light emitting device (LED) treatment to a wound of a patient comprising:
a plurality of LED modules, each LED module comprising at least one LED, wherein each LED module allows for consecutive abutment with another LED module; and
a controller coupled to the plurality of LED modules and controlling operation of each LED of the plurality of LED modules.

12. The system of claim 11, further comprising an electrical connector strip that couples the controller to the plurality of LED modules,
wherein each of the plurality of LED modules further comprises one or more electrical terminals, wherein the electrical connector strip attaches to each LED module such that electrical connection is made to the one or more electrical terminals.

13. The system of claim 11, wherein each of the plurality of LED modules further comprises a wireless communication device for coupling with the controller.

14. The system of claim 11, further comprising one or more sensors coupled to the controller.

15. The system of claim 14, wherein at least one of the one or more sensors is embedded in each of the plurality of LED modules.

16. The system of claim 14, wherein the one or more sensors comprises a skin-contact sensor, a light sensor, a temperature sensor, or a combination thereof.

17. The system of claim 11, wherein the at least one LED of each of the plurality of LED modules comprises an LED that emits a wavelength of about 380 to 470 nanometers.

18. The system of claim 11, wherein the controller is configured to operate any LED modules coupled thereto for delivering a specified dose of LED light for a predetermined period of time.

19. The system of claim 18, wherein the controller is further configured to interrupt or adjust the specified dose of LED light or the predetermined period of time according to feedback from one or more sensors.
